(19) United States
(12) Patent Application Publication
(10) Pub. No.: US 2019/0388702 A1
(43) Pub. Date: Dec. 26, 2019

(54) TUMOR TREATMENT METHOD

(71) Applicant: OLYMPUS CORPORATION, Tokyo (JP)

(72) Inventors: Toshiaki WATANABE, Tokyo (JP); Masahiro YOSHINO, Tokyo (JP); Tetsuhide TAKEYAMA, Tokyo (JP); Motohiro MITAMURA, Tokyo (JP)

(73) Assignee: OLYMPUS CORPORATION, Tokyo (JP)

(21) Appl. No.: 16/012,827

(22) Filed: Jun. 20, 2018

(51) Int. Cl. A61N 5/06; A61K 47/68; A61B 1/018; A61B 18/00

(52) U.S. Cl. CPC A61N 5/062; A61K 47/6803; A61N 5/0601; A61N 5/0603; A61B 1/018; A61N 2005/067; A61N 2005/0612; A61N 2005/0663; A61N 2005/061; A61N 2005/0651; A61B 18/00

(57) ABSTRACT

A tumor treatment method for treating a tumor in a subject, the method includes: a step I of administering a therapeutically effective amount of one or a plurality of antibody-IR 700 molecules to the subject, in which the antibody specifically binds to a cell surface protein of the tumor; a step II of inserting an optical probe into the subject; a step III of applying light having a wavelength in a range from 660 nm to 740 nm from the optical probe, to supply energy of at least 1 J/cm² to at least a part of the tumor; a step IV of waiting for a time until an immune response is elicited in the tumor; a step V of inserting an energy device into the subject; and a step VI of resecting tissues of the subject including the tumor using the energy device.

### BACKGROUND OF THE INVENTION

### 1. Field of the Invention

The present invention relates to a tumor treatment method using photoimmunotherapy.

### 2. Description of the Related Art

The photoimmunotherapy disclosed in U.S. Pat. No. 8,524,239 is known as a method for treating a tumor of human. The photoimmunotherapy induces death to a tumor by necrosis without killing normal cells around the tumor, to thereby be capable of treating the tumor.

### SUMMARY OF THE INVENTION

A tumor treatment method according to the present invention is a procedure for treating a tumor in a subject and the procedure includes: a step I of administering a therapeutically effective amount of one or a plurality of antibody-IR 700 molecules to the subject, in which the antibody specifically binds to a cell surface protein of the tumor; a step II of inserting an optical probe into the subject; a step III of applying light having a wavelength in a range from 660 nm to 740 nm from the optical probe to at least a part of the tumor, to supply energy of at least 1J/cm² to at least the part of the tumor; a step IV of waiting for a time until an immune response is elicited in the tumor; a step V of inserting an energy device into the subject; and a step VI of resecting tissues of the subject including the tumor using the energy device.

### BRIEF DESCRIPTION OF THE DRAWINGS

FIG. 1 is a flowchart showing a procedure of a tumor treatment method according to a first embodiment.

FIG. 2 illustrates a schematic configuration of an optical probe and an endoscope.

FIG. 3 is a flowchart showing a procedure of a tumor treatment method according to a second embodiment.

### DETAILED DESCRIPTION OF THE PREFERRED EMBODIMENTS

Hereinafter, preferred embodiments of the present invention will be described with reference to drawings. Note that, in each of the drawings used for the description below, a different scale size is used for each of the components in order to allow each of the components to be illustrated in a recognizable size in the drawings, and the present invention is not limited to the number, shapes, ratio of the sizes of the components, and a relative positional relationship among the components shown in these drawings.

### FIRST EMBODIMENT

The tumor treatment method according to the present embodiment is to treat a tumor in a subject which is a human body. More specifically, the tumor treatment method according to the present embodiment is used for treating a tumor generated in a lumen such as a digestive organ or a bladder in a human body. The tumor in the present embodiment is a gastrointestinal cancer, as one example.

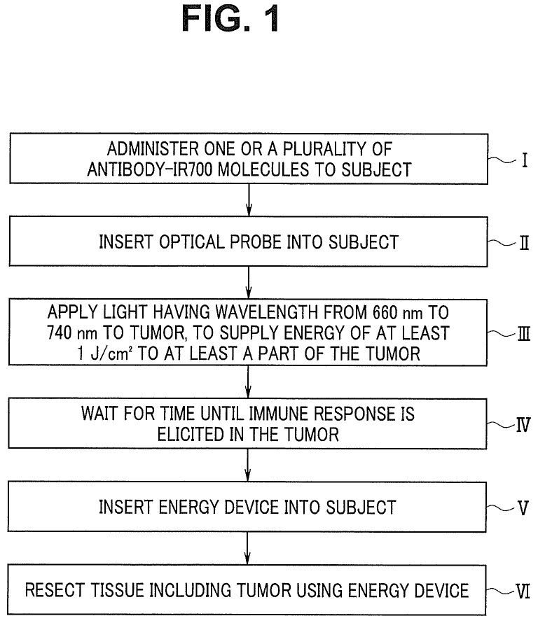

FIG. 1 is a flowchart showing a procedure of the tumor treatment method according to the present embodiment. In the tumor treatment method according to the present embodiment, Photoimmunotherapy (PIT) is used in a part of the procedure. The photoimmunotherapy is disclosed in the U.S. Pat. No. 8,524,239, for example. In the photoimmunotherapy, one or a plurality of antibody-IR700 molecules specifically binding to a cell surface protein of a tumor and light having a wavelength in a range from 660 nm to 740 nm are used. In the photoimmunotherapy, the antibody-IR700 molecules are brought into contact with tumor cells, and thereafter the light having the wavelength in the range from 660 nm to 740 nm is applied to at least a part of the tumor, to supply energy of at least 1J/cm² to at least the part of the tumor. The application of the light causes a cell death by the necrosis of the tumor cells to which the one or the plurality of antibody-IR700 molecules bind. Since a detail of the photoimmunotherapy is publicly known, description of the photoimmunotherapy will be omitted.

In the tumor treatment method according to the present embodiment, first, a therapeutically effective amount of one or a plurality of antibody-IR700 molecules are administered to the subject in step I. The one or the plurality of antibody-IR700 molecules are used for the above-described photoimmunotherapy. The one or the plurality of antibody-IR700 molecules can be administered to the subject by intravenous injection, for example. By executing the step I, the one or the plurality of antibody-IR700 molecules bind to the cell surface protein of the tumor.

Next, in step II, an optical probe is inserted into the subject. The optical probe is an apparatus configured to emit the light having the wavelength in the range from 660 nm to 740 nm in the subject.

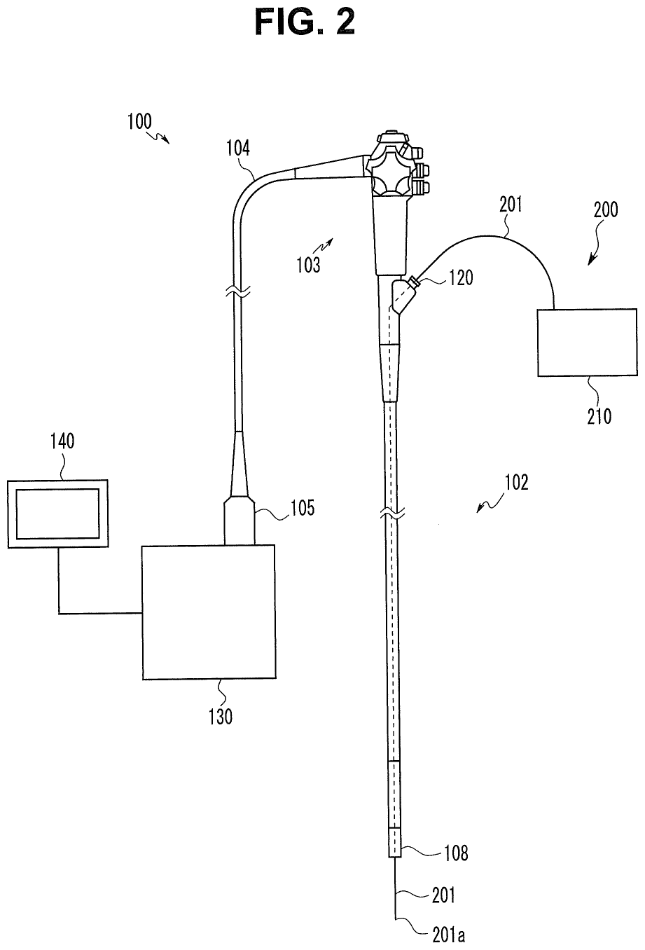

FIG. 2 illustrates one example of the configuration of the optical probe. An optical probe 200 shown in FIG. 2 includes a light source apparatus 210 and an elongated insertion portion 201 configured to be insertable into a subject. The light source apparatus 201 includes a laser diode or a light-emitting diode that emits light having the wavelength in the range from 660 nm to 740 nm.

The proximal end of the insertion portion 201 is connected to the light source apparatus 210. An optical fiber cable is inserted in the insertion portion 201. The light having the wavelength in the range from 660 nm to 740 nm, which is emitted from the light source apparatus 210, is emitted from a light-emitting window 201a provided at the distal end of the insertion portion 201, via the optical fiber cable.

Next, in the step II, a light-emitting portion of the optical probe 200 is placed in the vicinity of the tumor in the subject. That is, the light-emitting window 201a of the insertion portion 201 is placed in the vicinity of the tumor in the step II.

The method for inserting the insertion portion 201 of the optical probe 200 into the subject is not specifically limited. In the present embodiment, as one example, the insertion portion 201 of the optical probe 200 is inserted into the subject together with the endoscope 100 as shown in FIG. 2.

Since the configuration of the endoscope 100 is publicly known, detailed description of the configuration will be omitted. The endoscope 100 includes an elongated insertion portion 102 configured to be introduceable into a subject, and a configuration for observing the inside of the subject is provided at the insertion portion 102. Note that the endoscope 100 may be, what is called, a flexible endoscope configured such that the insertion portion 102 has flexibility, or may be, what is called, a rigid endoscope configured such that the insertion portion 102 does not have flexibility.

The endoscope 100 according to the present embodiment mainly includes the insertion portion 102, an operation portion 103 located on the proximal end of the insertion portion 102, and a universal cord 104 extended from the operation portion 103.

The distal end portion 108 of the insertion portion 102 includes a configuration for optically observing the inside of the subject. For example, the distal end portion 108 is provided with an image pickup unit which includes an objective lens and an image pickup device and which is configured to optically observe the inside of the subject. In addition, the distal end portion 108 is also provided with an illumination light emitting portion that emits light for illuminating an object of the image pickup unit, though not shown.

One end of a treatment instrument port 120 is open on the operation portion 103 disposed on the proximal end of the insertion portion 102. The treatment instrument port 120 is a conduit provided in the insertion portion 102. The other end of the treatment instrument port 120 is open at the distal end portion 108 of the insertion portion 102.

The treatment instrument port 102 allows the insertion portion 201 of the optical probe 200 to insert therein. As shown in FIG. 2, the insertion portion 201 of the optical probe 200 inserted from the one end of the treatment instrument port 120 is protruded from the distal end portion 108 of the insertion portion 102 of the endoscope 100.

The universal cord 104 is provided with an endoscope connector 105 configured to be connectable to an external apparatus 130. The external apparatus 130 causes an optical image picked up by the image pickup unit provided in the distal end portion 108 to be displayed on an image display apparatus. In addition, the external apparatus 130 includes a light source apparatus that generates illumination light.

In the tumor treatment method according to the present embodiment, first, the insertion portion 102 of the endoscope 100 is inserted into the subject, to place the distal end portion 108 in the vicinity of the tumor in the step II. Next, the insertion portion 201 of the optical probe 200 is inserted into the subject through the treatment instrument port 120.

Note that if the endoscope 100 is configured to be capable of emitting the light having the wavelength in the range from 660 nm to 740 nm from the distal end portion 108 of the insertion portion 102, the endoscope 100 is allowed to serve also as the optical probe 200.

Next, in step III, the light having the wavelength in the range from 660 nm to 740 nm is applied from the optical probe to a part of the tumor, to thereby supply energy of at least 1J/cm² to the part of the tumor. In the present embodiment, in the step III, the light-emitting window 201a of the optical probe 200 is directed to the tumor under the observation with the endoscope 100, to apply the light to the tumor. When the application of the above-described dose of light to the tumor is finished, the optical probe 200 is extracted from the subject.

Next, in step IV, an operator waits for a time until immune response is elicited in the tumor. That is, after the occurrence of the cell death by the necrosis of the tumor cells in the step III, the operator waits for the time until immune cells gather and the immune response is elicited in the tumor in the step IV. In the step IV, the insertion portion 102 of the endoscope 100 inserted in the subject in the step III may be extracted or remained inserted in the subject. In the present embodiment, as one example, the insertion portion 102 of the endoscope 100 is extracted from the inside of the subject in the step IV.

Next, in step V, an energy device is inserted into the subject. The energy device is a publicly known device for resecting tissues in the subject. The energy device is an electrocautery scalpel, an ultrasound scalpel, or a laser scalpel, or a combination of these scalpels, for example.

The method for inserting the energy device into the subject is not specifically limited. In the present embodiment, as one example, the energy device is inserted into the subject together with the endoscope 100. As the energy device, an elongated electrocautery scalpel having a loop-shaped distal end is used, for example. That is, in the present embodiment, the energy device is inserted into the subject through the treatment instrument port 120 of the endoscope 100.

Note that the endoscope 100 to be used in the step V may be the same one as the endoscope 100 used in the step II, or may be an endoscope different from the endoscope 100. That is, if the endoscope 100 is not extracted from the inside of the subject in the step IV, the endoscope inserted into the subject in the step II can be used as-is in the step V.

Next, in step VI, the tissues including the tumor of the subject are resected using the energy device, and the resected tissues are collected. Note that the entirety of the tumor (called tumor for the sake of convenience, though the tumor cells have already been killed if the sufficient amount of optical energy is applied to the entirety of the tumor at the time of execution of the step III) and the normal tissues around the tumor are resected in the step VI.

In the present embodiment, the resection of the tissues using the energy device is performed under the observation with the endoscope 100. In the step VI, a resection method called endoscopic mucosal resection (EMR) is used, for example.

As described above, in the tumor treatment method according to the present embodiment, a part of the tumor in the subject is killed by the photoimmunotherapy, and thereafter the tumor and the normal tissues around the tumor are resected in the step VI. Therefore, the tumor treatment method according to the present embodiment produces a therapeutic effect on a lesion other than the tumor by the immune cells activated in the step IV, and makes it possible to surely determine whether or not the tumor is completely resected by performing pathological examination for determining presence or absence of the tumor cells on the cut surface of the tissues resected in the step VI.

In the description above, the case where the tumor treatment method is used for treating the gastrointestinal cancer has been described. However, the tumor treatment method according to the present embodiment can be used for treating a bladder cancer. If the tumor treatment method according to the present embodiment is used for treating the bladder cancer, the endoscope to be inserted into the subject in the step II and the step V is a type, what is called, a resectscope. In this case, the optical probe, the energy device, and the endoscope are inserted into the subject through the urethra. In addition, in this case, an energized small loop-shaped wire electrode is connected as the energy device, and a resection method called transurethral resection of the bladder tumor (TURBT) is used, for example, for resecting the tissues in the step VI.

### SECOND EMBODIMENT

Hereinafter, description will be made on the second embodiment of the present invention. Only the points different from the first embodiment will be described below. The same constituent elements as those in the first embodiment are attached with the same reference numerals and descriptions thereof will be omitted as needed.

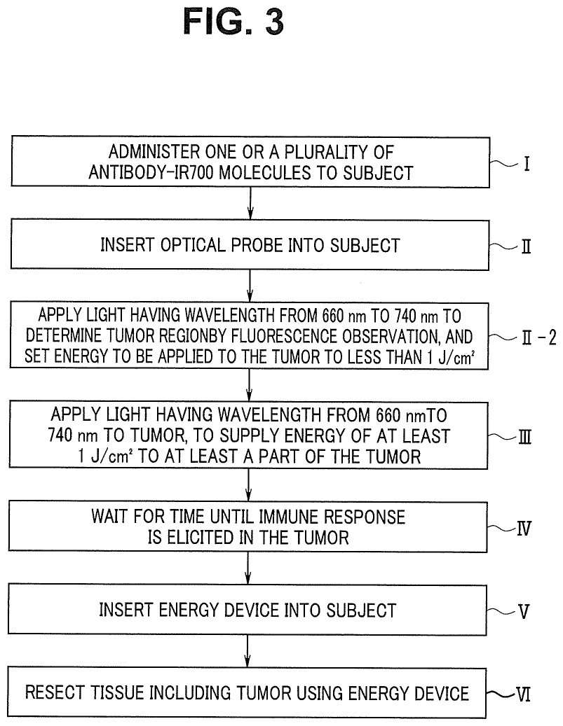

FIG. 3 is a flowchart showing a procedure of a tumor treatment method according to the present embodiment. The tumor treatment method according to the present embodiment shown in FIG. 3 is different from the first embodiment in that a step II-2 is included between the step II and the step III.

In the step II-2, the light having the wavelength in the range from 660 nm to 740 nm is applied from the optical probe to a part where a tumor exists such that the amount of energy is less than 1J/cm². When the energy of less than 1J/cm² is applied to the antibody-IR700 molecules binding to the tumor cells, the cell death by necrosis does not occur in the tumor cells. Therefore, in the step II-2, the IR 700 molecules binding to the tumor cells emit fluorescence. Note that if the endoscope 100 is configured to be capable of emitting the light having the wavelength in the range from 660 nm to 740 nm from the distal end portion 108 of the insertion portion 102, the endoscope 100 is allowed to serve also as the optical probe 200.

Then, in the step II-2, the fluorescence emitted from the IR 700 molecules is observed with the endoscope 100 in the step II-2. The part where the fluorescence is observed is the part where the tumor cells to which the antibody-IR700 molecules bind exist. Therefore, in the step II-2, the part where the fluorescence is observed in the subject is determined to be a region of the tumor. Then, the region of the tumor determined in the step II-2 is marked by dyeing of the tissues, for example, to bring the region of the tumor into an observable state in the subsequent steps III and VI.

The tumor treatment method according to the present embodiment enables the region where the tumor exists in the subject to be surely recognized, to thereby enable the application of the light in the step III and the resection of the tissues in the step VI to be properly performed.

Note that the endoscope 100 used in the present embodiment includes a configuration for observing the fluorescence emitted from the IR 700 molecules. Specifically, the image pickup unit included in the endoscope 100 includes a filter configured to cut the light having the wavelength in the range from 660 nm to 740 nm, which is emitted from the optical probe. That is, the image pickup unit includes a filter configured to cut the light having the wavelength that excites the IR 700 molecules. The image pickup unit with such a filter is capable of observing the fluorescence emitted from the IR 700 molecules. Note that the filter may be capable of being removed from the optical axis of the image pickup unit by remote control.

Note that the present invention is not limited to the above-described embodiments and it is needless to say that various modifications and applications are possible in a range without departing from the gist of the invention. Furthermore, the above-described embodiments include the invention at various stages, and various inventions can be extracted by appropriately combining a plurality of components disclosed.

What is claimed is:

1. A tumor treatment method for treating a tumor in a subject, the method comprising:
a step I of administering a therapeutically effective amount of one or a plurality of antibody-IR 700 molecules to the subject, in which the antibody specifically binds to a cell surface protein of the tumor;
a step II of inserting an optical probe into the subject;
a step III of applying light having a wavelength in a range from 660 nm to 740 nm from the optical probe to at least a part of the tumor, to supply energy of at least 1J/cm² to at least the part of the tumor;
a step IV of waiting for a time until an immune response is elicited in the tumor;
a step V of inserting an energy device into the subject; and
a step VI of resecting tissues of the subject including the tumor using the energy device.

2. The tumor treatment method according to claim 1, wherein
in the step II, the optical probe is inserted into the subject together with a first endoscope,
in the step III, the light is applied under an observation with the first endoscope,
in the step V, the energy device is inserted into the subject together with a second endoscope, the second endoscope being an endoscope same as or different from the first endoscope, and
in the step VI, the tissues are resected under an observation with the second endoscope.

3. The tumor treatment method according to claim 1, wherein
the tumor is a gastrointestinal cancer,
in the step II, the optical probe is inserted into the subject through a treatment instrument port of the first endoscope inserted in the subject,
in the step III, the light is applied under an observation with the first endoscope, and the optical probe is extracted from the treatment instrument port after the application of the light,
in the step V, the energy device is inserted into the subject through a treatment instrument port of the second endoscope, the second endoscope being an endoscope same as or different from the first endoscope, and
in the step VI, the tissues are resected under an observation with the second endoscope.

4. The tumor treatment method according to claim 1, wherein
the tumor is a bladder cancer,
in the step II, the optical probe is inserted into the subject through a urethra, and
in the step V, the energy device is inserted into the subject through the urethra.

5. The tumor treatment method according to claim 1, further comprising, between the step II and the step III, a step 11-2 of applying the light having the wavelength in the range from 660 nm to 740 nm from the optical probe and observing fluorescence with energy of less than 1J/cm², to thereby determine a region of the tumor.
